(19) United States
(12) Patent Application Publication
(10) Pub. No.: US 2025/0366752 A1
(43) Pub. Date: Dec. 4, 2025

(54) ELECTRODE FOR ATTACHMENT TO THE HUMAN SKIN

(71) Applicant: Leonhard Lang GmbH, Innsbruck (AT)

(72) Inventors: Burrhus LANG, Innsbruck (AT); Daniel Fischer, Völs (DE)

(21) Appl. No.: 19/301,542

(22) Filed: Aug. 15, 2025

Related U.S. Application Data
(63) Continuation of application No. PCT/AT2024/060053, filed on Feb. 15, 2024.

(30) Foreign Application Priority Data
Feb. 17, 2023 (AT) A50109/2023

(51) Int. Cl. A61B 5/257; A61B 5/265; A61B 5/273

(52) U.S. Cl. CPC A61B 5/257; A61B 5/273; A61B 5/265; A61B 2562/125

(57) ABSTRACT

An electrode for attachment to the human skin includes an electrically nonconductive carrier and an electrically conductive connection element for connecting a signal conductor. A conductor is at least partially arranged on the lower side of the carrier intended to be skin side, and is electrically connected to the connection element and to a contact medium intended to be skin side. The connection element is joined to the signal conductor and/or the conductor by heat staking.

The present application is a continuation of International Application PCT/AT2024/060053 filed on Feb. 15, 2024. Thus, all of the subject matter of International Application PCT/AT2024/0600653 is incorporated herein by reference.

### BACKGROUND OF THE INVENTION

The invention relates to an electrode for attachment to the human skin, comprising an electrically nonconductive carrier, and an electrically conductive connection element for connecting a signal conductor. A conductor is arranged at least partially on the lower side of the carrier intended to be facing the skin, and is electrically connected to the connection element and to a contact medium intended to be facing the skin. The invention also relates to a method for producing an electrode for attachment to the human skin.

Such electrodes are already known from the prior art. In this case, the conductor is usually a metal foil. The signal conductor is then riveted to the conductor and the carrier to create an electrical and a mechanical connection between the conductor/carrier and the signal conductor. The disadvantage is that riveting the signal conductor to the conductor/carrier is relatively complex.

When performing exams using various X-ray techniques, it is often desirable to collect additional data or monitor certain patient parameters using measuring electrodes during the exams. However, the electrodes described above are not suitable for this purpose because the electrodes or metallic components create shadows on the X-ray image, which can make the X-ray image unsuitable for further analysis.

To avoid this, it is known to manufacture a conductor from X-ray transparent materials, such as carbon fiber or conductive plastic, in order to minimize the influence of the electrodes on an X-ray image. However, metallic conductors and connection elements are often still used, which in turn are visible on an X-ray image.

If an X-ray transparent signal conductor is also provided, this is usually glued to the conductor. To do this, the wires of the signal conductor are fanned out and glued to the conductor using adhesive and/or adhesive tape. The purpose of fanning out is to create the best possible electrically conductive connection between the signal conductor and the conductor. This type of connection is complex and has relatively low mechanical strength.

### SUMMARY OF THE INVENTION

The object of the invention is to at least partially remedy the disadvantages described above and to provide an electrode which is improved compared to the prior art and a method for producing an electrode.

According to the invention, it is therefore provided that the connection element is heat staked to the signal conductor and/or the conductor.

Heat staking represents a simple and uncomplicated way to electrically and mechanically connect the signal conductor to the connection element and/or the conductor to the connection element and thus the signal conductor via the connection element to the conductor. The heat staking creates a positive and force-fitting connection, so that sufficient mechanical strength can be guaranteed.

With regard to a method according to the invention, the following method steps are provided:
arranging, preferably by gluing or printing, a conductor on the lower side of an electrically non-conductive carrier intended to be facing the skin,
inserting a connection element into the carrier, wherein the connection element—preferably with a flange-like holding region—rests against the lower side of the conductor to be facing the skin and/or the upper side of the carrier facing away from the skin,
inserting a signal line into a receiving region of the connection element, and
heat staking the receiving region.

Compared to gluing or riveting the signal conductor to the conductor/carrier, heat staking is much easier and faster to perform. This allows an electrode to be manufactured in a shorter time, which in turn brings economic advantages.

Particularly preferably, the electrode is at least partially X-ray transparent. This makes it possible to use the electrode for measurements during X-ray examinations.

Preferably, the connection element is made of a conductive plastic and/or carbon fiber.

It may further be provided that the conductor is made of a conductive plastic and/or carbon fiber.

Finally, the signal conductor is at least partially made of a conductive plastic and/or carbon fiber.

This represents a simple way to make the conductive components of the electrode X-ray transparent. In particular, it is advantageous if the connection element, the conductor and the signal conductor are made of a conductive plastic and/or carbon fiber, since the electrode is thus completely X-ray transparent.

The conductor can also be coated on its side facing the contact medium with a pair of silver/silver chloride, tin/tin chloride or another redox pair suitable, for example, for depolarizing the electrode.

These redox pairs are used to achieve low noise and depolarization in an electrode in the event of defibrillation. These can be oxidized or reduced and in the process absorb or release at least one electron. The most commonly used are silver/silver chloride, tin/tin chloride and tin-antimony. However, for the present invention, all redox pairs are conceivable which enable depolarization of the electrode. The redox pairs can be actively added or possibly generated in situ by reactions.

The signal conductor can have at least one wire and insulation.

The at least one wire and/or the insulation can be heat staked to the connection element.

The wire can be used to create an electrical and mechanical connection between the signal conductor and the connection element. If the insulation is also heat staked to the connection element, a more stable mechanical connection can be achieved.

According to one exemplary embodiment, the at least one wire and the insulation can be heat staked separately from one another to the connection element. This allows the insulation to act as strain relief for the signal conductor.

In principle, it is also conceivable that the connection element or a part of the connection element is an integral part of the signal conductor. For example, the connection element or a part thereof can be injection molded onto the signal conductor during production.

The connection element can also be formed in one piece. This represents the simplest and most cost-effective design of a connection element. Of course, a multi-part design of the connection element is also conceivable.

Advantageously, the connection element can have at least one flange-like holding region for contact with the lower side of the conductor to be facing the skin and/or the upper side of the carrier facing away from the skin.

This facilitates the production of an electrode, as the connection element can be fixed to the electrode via the holding region. In addition, the holding region of the electrode provides increased stability in the region of connection to the signal conductor.

Preferably, the connection element can have at least one receiving region for at least partially receiving the signal line. The receiving region can be formed by a simple opening or by slots. In principle, however, various designs of a receiving region are conceivable.

The connection element can also have a connection region for connecting the connection element to the conductor and/or carrier.

The connection region can also take on a variety of forms. A simple option would be a pin, which is then heat staked.

If the carrier and/or the conductor has an opening for inserting the connection element, the connection element can be easily and securely connected to the carrier and/or conductor.

Furthermore, the connection element can be connected to the carrier by interposing the conductor on the lower side and the upper side of the carrier. This ensures the best possible mechanical connection between the connection element and the carrier.

Preferably, the carrier can be coated on the lower side intended to be facing the skin with adhesive, preferably a skin adhesive, which is preferably self-adhesive or thermo-activatable, or has a plaster layer provided with an adhesive, preferably a skin adhesive. This provides a simple way to position the electrode on a patient.

Particularly preferably, the adhesive can be electrically conductive and the contact medium can be formed by the adhesive. This simplifies the design and manufacture of the electrode.

With regard to a method, before the insertion of a connection element, a through opening can be produced through the carrier and the conductor, preferably by punching. The connection element can then be inserted into the opening.

A connection region—and thus the connection element with the carrier—can be heat staked. This provides a simple way to connect the connection element to the carrier.

The following additional steps may also be provided:
coating the lower side of the carrier to be facing the skin with adhesive, or
applying a plaster layer provided with an adhesive, preferably a skin adhesive, and
introducing a contact medium—preferably a gel—into a recess in the plaster layer so that the underlying conductor is contacted.

### BRIEF DESCRIPTION OF THE DRAWINGS

Further details and advantages of the invention will be explained in more detail below with reference to the drawings, in which:

FIG. 1 is a schematic bottom view (later the side facing the skin) of the manufacturing steps of an embodiment of an electrode,

FIG. 2 is a schematic plan view of the manufacturing steps of an embodiment of an electrode, wherein only a part of the process steps is shown in a plan view,

FIG. 3 is a schematic representation of the sequence of sections along the line A-A of FIG. 1,

FIG. 4 is a schematic bottom view (later the side facing the skin) of the manufacturing steps of an exemplary embodiment of an electrode,

FIG. 5 is a schematic plan view of the manufacturing steps of a further exemplary embodiment of an electrode, wherein only a part of the process steps is shown in a plan view,

FIG. 6 is a schematic representation of the sequence of sections along the line A-A of FIG. 4, and

FIGS. 7a-7e are schematic representations of different embodiments of a connection element.

### DETAILED DESCRIPTION OF THE INVENTION

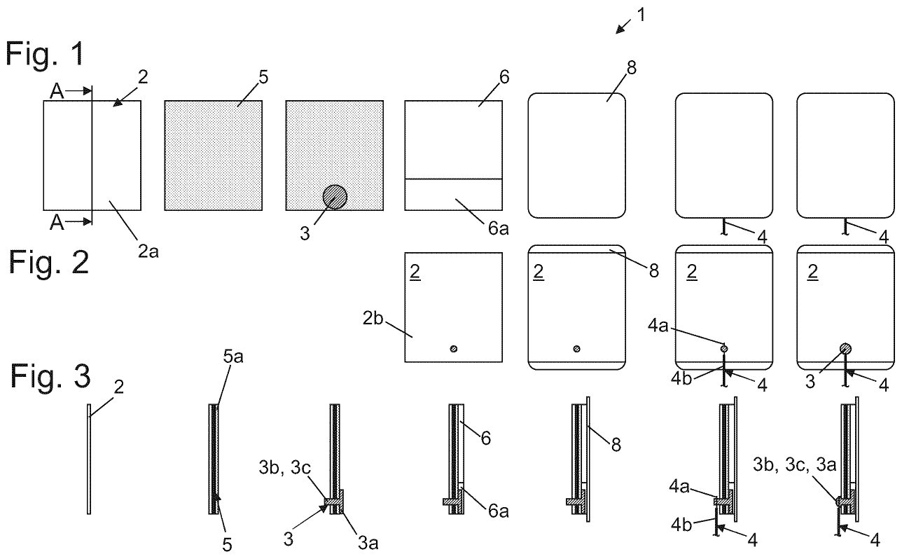

With reference to FIGS. 1 to 3, the method sequence for producing an exemplary embodiment of an electrode 1 according to the invention for attachment to the human skin will be explained in more detail below.

The starting point is an electrically nonconductive carrier 2. The carrier material serves to anchor the electrical components of the electrode 1. It can, for example, consist of a (flexible) film (e.g. PET or TPU).

In a next step, a conductor 5 is applied to this carrier material, which in this exemplary embodiment completely covers the carrier 2. It is also conceivable that the conductor 5 is applied only at certain points on the carrier 2. In particular, conductor 5 is glued or printed.

In this exemplary embodiment, the conductor 5 consists of carbon fiber or conductive plastic. If X-ray transparency is not desired, conductor 5 could also be made of metal.

In this exemplary embodiment, the conductor 5 is coated with a layer 5a of, for example, silver/silver chloride, tin/tin chloride, tin-antimony or another redox pair.

In a further step, an opening 7 is now provided through the electrical conductor 5 and the carrier 2. This can be done, for example, by punching. This is followed by the insertion of the connection element 3.

In the illustrated exemplary embodiment, the connection element 3 has a cylindrical section, to which a flange-shaped, laterally projecting holding region 3a is connected. In this exemplary embodiment, both the receiving region 3b and the connection region 3c are formed on or through the cylindrical portion. Overall, the laterally projecting flange-shaped holding region 3a is essentially plate-shaped.

If a connection element 3 is used which consists of a single part, a cost-effective production of the electrode is possible. For mechanical anchoring, the one-piece design of the connection element 3 is sufficient.

In the next step, a coating of biocompatible adhesive is applied to the lower side 2a of the carrier 2, by means of which adhesive the electrode 1 can be fixed to the skin of a patient.

In this exemplary embodiment, the biocompatible adhesive is electrically conductive and thus acts as a contact medium 6. The electrical contact medium 6 enables the (preferably ion-based) conduction of body-generated electrical potentials or device-generated measuring or stimulation currents from the body surface (skin) to the electrical connection element 3 and vice versa.

Alternatively, it is also conceivable that a plaster layer is applied, which serves to fix the electrode 1 on a patient's skin. The contact medium 6 can then consist, for example, of a gel doped with chlorides, which is present either in a more or less liquid form (more or less gelled) or as a cross-linked polymer matrix (hydrogel).

Suitable plaster materials can consist, for example, of a film (e.g. PE), a foam tape (e.g. PE foam) or nonwovens. The plaster materials are usually coated with a biocompatible adhesive on the patient side.

In addition, a rivet insulation 6a is applied, which insulates the region in which the connection element 3 is arranged from the skin of a patient. This can prevent excessive stress on a patient's skin below the connection element 3.

In a further manufacturing step of the electrode according to FIGS. 1 to 3, a cover 8 is applied, which protects the contact medium 6 until the electrode 1 is used.

In a next step, a signal conductor 4, in this embodiment only the wires 4a, is introduced into a receiving region 3b of the connection element 3. The receiving region has an opening into which the signal conductor 4 can be inserted.

In order to definitely fix the electrical connection element 3 in the electrode 1 and to establish an electrically conductive connection between the signal conductor 4 and the conductor 5, in a next step the signal conductor 4 is heat staked in the receiving region 3b to the connection element 3 and the carrier 2 and the conductor 5 are heat staked in the connection region 3c to the connection element 3.

Heat staking can be carried out in the traditional way using a heated stamp or contactlessly using laser or infrared radiation.

Since in this exemplary embodiment the receiving region 3b and the connection region 3c are combined, heat staking only needs to be carried out once. This allows an electrode 1 to be produced quickly and easily. The heat staking creates a rivet head, which in turn acts as a holding region 3a.

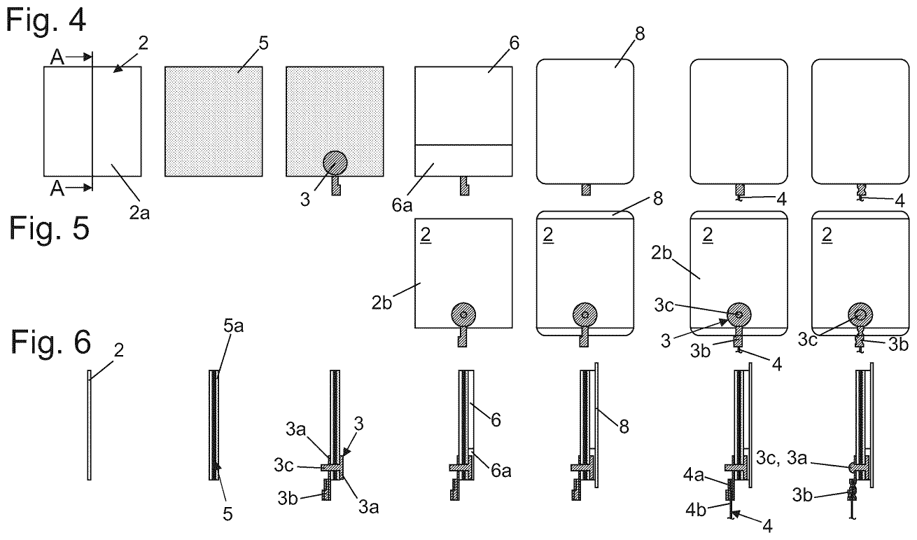

In the exemplary embodiment shown in FIGS. 4 to 6, most of the method steps correspond to those in FIGS. 1 to 3, which is why the same reference numerals designate the same parts. The difference essentially consists in the use of a different embodiment of a connection element 3.

It can be seen that the connection element 3 is now formed in two parts. A lower part is similar to the connection element 3 according to FIGS. 1 to 3 and has a holding region 3a and a cylindrical section. The upper part also has a holding region 3a, wherein the upper holding region 3a has an opening for receiving the cylindrical section. The cylindrical section together with the corresponding opening forms the connection region 3c.

In the upper part, there is also shown a section that resembles a wire end ferrule. This section forms the receiving region 3b.

In this exemplary embodiment, repeated heat staking is necessary. First, the connection region 3c is heat staked to the carrier 2 and the conductor 5 in order to establish a mechanical connection between the connection element and the carrier as well as an electrical connection between the connection element 3 and the conductor 5.

Furthermore, the receiving region 3b of the connection element 3 is heat staked to the signal conductor 4. The wires 4a and the insulation 4b are heat staked separately from one another to the connection element 3. By separately connecting the wires 4a and insulation 4b, strain relief of the signal conductor 4 can be achieved.

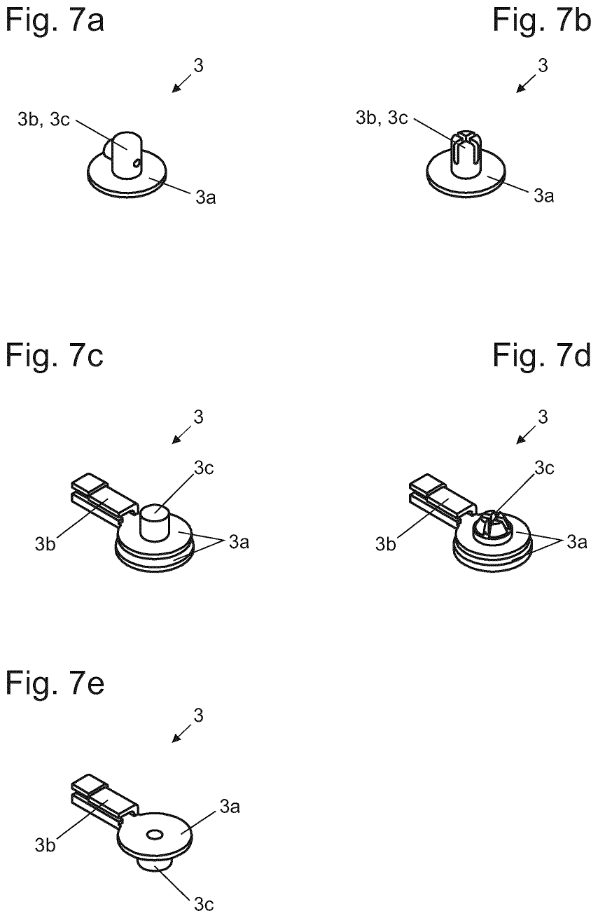

FIGS. 7a to 7e show schematic representations of various embodiments of a connection element 3.

The exemplary embodiment according to FIG. 7a is similar to the connection element 3 shown in FIGS. 1 to 3. In contrast, the connection element 3 according to FIG. 7a has an additional extension extending the opening for receiving the signal conductor 4. This extension serves to accommodate the insulation 4b of the signal conductor 4, whereby additional mechanical stability can be achieved in the connection between the signal conductor 4 and the connection element 3.

The embodiment according to FIG. 7b has slots for receiving the signal conductor 4, into which slots the wires 4a of the signal conductor 4 can be inserted. In this exemplary embodiment too, the receiving region 3b and the connection region 3c are combined.

The exemplary embodiment according to FIG. 7c corresponds to the exemplary embodiment shown in FIGS. 4 to 6. The embodiment according to FIG. 7d is very similar to that of FIG. 7c, but has a snap connector in the connection region 3b for connecting the connection element 3 to the carrier 2 and conductor 5. In this exemplary embodiment, only the signal conductor 4 is thus heat staked to the connection element 3.

The connection element 3 according to FIG. 7e is a further modification of the exemplary embodiments according to FIGS. 7c and 7d. The connection element 3 is again designed in one piece and has only one holding region 3a. A cylindrical section adjoins the holding region 3a in a downward direction. The connection element according to FIG. 7e is not inserted into the carrier 2 from the lower side 2a, but from the upper side 2b. The connection element 3 can be connected to the carrier 2 and the conductor 5 by heat staking the cylindrical section. By forming a rivet head during heat staking, a holding region 3a is formed on the lower side 2a of the carrier 2, which also establishes the electrical contact to the conductor.

The exemplary embodiments according to FIGS. 7a to 7e only provide examples of different possibilities for the design of connection element 3. Of course, a variety of other designs of connection elements are also conceivable within the scope of this invention.

### LIST OF REFERENCE NUMERALS

1 electrode
2 carrier
2a lower side
2b upper side
3 connection element
3a holding region
3b receiving region
3c connection region
4 signal conductor
4a wire
4b insulation
5 conductor
5a coating redox pair
6 contact medium
6a rivet insulation
7 opening
8 cover

1. An electrode for attachment to the human skin, comprising:
an electrically nonconductive carrier,
an electrically conductive connection element for connecting a signal conductor, and
a conductor arranged at least partially on the lower side of the carrier intended to be facing the skin and electrically connected to the connection element and to a contact medium intended to be facing the skin,
wherein the connection element is heat staked to the signal conductor and/or the conductor.

2. The electrode according to claim 1, wherein the electrode is at least partially X-ray transparent.

3. The electrode according to claim 1, wherein the connection element is made of a conductive plastic and/or carbon fiber.

4. The electrode according to claim 1, wherein the conductor is made of a conductive plastic and/or carbon fiber.

5. The electrode according to claim 1, wherein the signal conductor is at least partially made of a conductive plastic and/or carbon fiber.

6. The electrode according to claim 1, wherein the conductor is coated on its side facing the contact medium with a pair of silver/silver chloride, tin/tin chloride or another redox pair suitable, for example, for depolarizing the electrode.

7. The electrode according to claim 1, wherein the signal conductor has at least one wire and an insulation.

8. The electrode according to claim 7, wherein the at least one wire and/or the insulation is heat staked to the connection element.

9. The electrode according to claim 7, wherein the at least one wire and the insulation are heat staked separately from one another to the connection element.

10. The electrode according to claim 1, wherein the connection element is comprised of a single part.

11. The electrode according to claim 1, wherein the connection element has at least one flange-like holding region for contact with the lower side of the conductor intended to be facing the skin and/or the upper side of the carrier facing away from the skin.

12. The electrode according to claim 1, wherein the connection element has at least one receiving region for at least partially receiving the signal line.

13. The electrode according to claim 1, wherein the connection element has a connection region for connecting the connection element to the conductor and/or carrier.

14. The electrode according to claim 1, wherein the carrier and/or the conductor has an opening for introducing the connection element.

15. The electrode according to claim 1, wherein the connection element is connected to the carrier on the lower side and the upper side of the carrier, the conductor being interposed between the connection element and the lower side of the carrier.

16. The electrode according to claim 1, wherein the carrier is coated with adhesive on the lower side to be facing the skin, preferably a skin adhesive, which is preferably self-adhesive or thermoactivatable, or has a plaster layer provided with an adhesive, preferably a skin adhesive.

17. The electrode according to claim 16, wherein the adhesive is electrically conductive and the contact medium is formed by the adhesive.

18. A method for producing an electrode for attachment to the human skin, in particular according to claim 1, comprising:
arranging, preferably by gluing or printing, a conductor on the lower side of an electrically nonconductive carrier intended to be facing the skin,
inserting a connection element into the carrier, wherein the connection element—preferably with a flange-like holding region—rests against the lower side of the conductor to be facing the skin and/or the upper side of the carrier facing away from the skin, and
inserting a signal line into a receiving region of the connection element, and heat staking the receiving region.

19. The method according to claim 18, wherein before the insertion of a connection element a through opening is produced through the carrier and the conductor, preferably by punching.

20. The method according to claim 18, wherein a connection region—and thus the connection element with the carrier—is heat staked.

21. The method according to claim 18, further comprising, before the insertion of the signal line:
coating the lower side of the carrier to be facing the skin with adhesive, or
applying a plaster layer provided with an adhesive, preferably a skin adhesive, and
introducing a contact medium—preferably a gel—into a recess in the plaster layer so that the underlying conductor is contacted.
